(19) United States
(12) Patent Application Publication
(10) Pub. No.: US 2023/0144166 A1
(43) Pub. Date: May 11, 2023

(54) SYSTEMS AND METHODS USED TO ENHANCE ARTIFICIAL INTELLIGENCE SYSTEMS BY MITIGATING HARMFUL ARTIFICIAL INTELLIGENCE ACTIONS

(71) Applicant: HI LLC, Los Angeles, CA (US)

(72) Inventors: Jamu Alford, Lake Arrowhead, CA (US); Patrick House, Seattle, WA (US); Gabriel Lerner, Los Angeles, CA (US); Ethan Pratt, Santa Clara, CA (US)

(73) Assignee: HI LLC, Los Angeles, CA (US)

(21) Appl. No.: 17/399,360

(22) Filed: Aug. 11, 2021

Related U.S. Application Data
(60) Provisional application No. 63/077,227, filed on Sep. 11, 2020.
(60) Provisional application No. 63/124,711, filed on Dec. 11, 2020.

(51) Int. Cl. G06N 20/00; G06N 5/022; G16H 40/63

(52) U.S. Cl. CPC G06N 20/00; G06N 5/022; G16H 40/63

(57) ABSTRACT

A system comprises memory configured for storing an emotional response engine configured for predicting an emotional state set in response to an input of a real-life scenario that may occur in the context of a range of use of an AI control system. The system further comprises user interfaces (UIs) configured for presenting the real-life scenario to human subjects. The system further comprises at least one non-invasive brain interface assembly configured for detecting brain activity of the human subjects in response to presenting the real-life scenario to each of the human subjects. The system further comprises a processor configured for determining a plurality of emotional state sets respectively for the human subjects based on the detected brain activity of the respective human subject, and updating the emotional response engine based on the predicted emotional state set and the determined emotional state sets.

### RELATED APPLICATION DATA

Pursuant to 35 U.S.C. § 119(e), this application claims the benefit of U.S. Provisional Application 63/077,227, filed Sep. 11, 2020 and U.S. Provisional Application 63/124,711, filed Dec. 11, 2020, which are expressly incorporated herein by reference.

### FIELD OF THE INVENTION

The present inventions relate to artificial intelligence, and in particular, methods and systems related to learning and predicting the behavior of a human to enhance artificial intelligence systems.

### BACKGROUND OF THE INVENTION

An artificial intelligence (AI) system is a computer system capable of stimulating human intelligence. Unlike an existing rule-based smart system, the AI control system is self-taught, makes decisions by itself, and gets smarter over time. When the AI control system is frequently used, the AI control system has an increased recognition rate and trains user preference more accurately. Thus, existing rule-based smart systems are gradually being replaced with AI control systems. AI technology includes machine learning (deep learning) and element technologies utilizing machine learning. Machine learning is an algorithm technique for self-classifying/learning characteristics of input data. An element technique is a technique of using a machine learning algorithm, such as deep learning, so as to copy functions, e.g., recognition, judgment, etc. of a human brain, and includes various technical fields, such as linguistic understanding, visual understanding, reasoning/prediction, knowledge representation, and motion control.

As AI control systems are used in ever increasing numbers of tasks and sophistication, they are running head-long into questions of “moral” behavior. Humans continuously make moral decisions and have been trained to do so from childhood. However, training AI control systems to make moral decisions is challenging. In the past, a simple AI control system playing Pong or running a spell check on a document did not need to be concerned with the morality of what it was doing; it simply tried to anticipate the location of a ball or guess what word the user is attempting to spell.

Today, however, more sophisticated AI-performing tasks, such as operating an autonomous car or providing medical diagnoses, require a more complex decision-making capability where it must choose from options that are not easily reduced to a decision tree or minimizing a penalty. In the near future, AI control systems may pilot planes and spaceships, design bridges, run national economies, decide medical treatments, and make military decisions. In these situations, the morality function, a component for determining the “best” decision, will be highly complex and may not be easily encoded in a decision tree coded by an engineer.

An AI control system may be posed with making a moral decision between multiple options in a crisis in a manner that mimics the best of human behavior. For example, when a driver slams on the brakes to avoid hitting a pedestrian crossing the road illegally, they are making a decision that shifts risk from the pedestrian to the people inside the car (see Amy Maxmen, “A Moral Map for AI Cars,” Nature, Vol. 562, 25 Oct. 2018, page 469)(https://www.nature.com/articles/d41586-018-07135-0). Thus, an autonomous car may be forced to decide to either save the driver or kill a pedestrian when posed with one situation or to decide to either hit a family pet or cause extensive property damage when posed with another situation. Autonomous cars might soon have to make such ethical judgements on their own. However, a survey of 2.3 million people around the world suggests that settling on a universal moral code for the autonomous vehicles could be a thorny task, since cultural differences affect moral decision making in humans (see Id.).

In contrast to selecting between multiple options in a crisis, an AI control system may purposely select what can be considered unethical strategies as necessary to optimize a task. In particular, modern AI is great at optimizing—finding the shortest route, the perfect pricing sweet spot, or the best distribution of a company's resources, but it's also blind cognizant of, particularly when it comes to ethics (see Edd Gent, “AI Behaving Badly: New Model Could Help AI Make More Ethical Choices,” (https://singularityhub.com/2020/07/06/ai-behaving-badly-new-model-could-help-ai-make-more-ethical-choices/) In fact, researchers showed that AI tasked with maximizing returns is actually disproportionally likely to pick an unethical strategy in fairly general conditions. (see Id.).

Irrespective of morality, an AI control system may be also posed with a decision of whether to demonstrate kindness or not. For example, consider an AI control system that is programmed to stop automobile traffic for pot-hole repair on a highway. Developing an AI-powered robot to detect pot-holes and stop traffic is relatively straightforward with current tools, and many programmers could easily write this function. However, if a mother duck with a string of ducklings was about to cross the highway, the AI-powered robot, if not expressly programmed to do so, will not stop the traffic and let the ducks pass safely. It is noted that, since the ducks are not yet on the highway, a simple “accident avoidance” algorithm would not have been sufficient.

There, thus, remains a need to incorporate a morality or kindness function into AI control systems, where such functions are designed to mitigate harmful AI actions.

### SUMMARY OF THE INVENTION

In accordance with a first aspect of the present inventions, a system for training an emotional response engine for use in an artificial intelligence (AI) system is provided. The system comprises memory configured for storing the emotional response engine. The emotional response engine is configured for predicting an emotional state set in response to an input of a real-life scenario that may occur in the context of a range of use of the AI control system. The system further comprises at least one user interface (UI) configured for presenting the real-life scenario to each of a plurality of human subjects, and at least one non-invasive brain interface assembly configured for detecting brain activity of the human subjects in response to presenting the real-life scenario to each of the human subjects.

The system further comprises at least one processor configured for determining a plurality of emotional state sets respectively for the human subjects based on the detected brain activity of the respective human subject, and updating the emotional response engine based on the predicted emotional state set and the determined emotional state sets. In one embodiment, the processor(s) is configured for reducing the determined emotional state sets into a single reference emotional state set representative of a collective emotional response of the human subjects, comparing the single reference emotional state set and the predicted emotional state set, generating at least one error based on the comparison, and updating the emotional response engine based on the error(s).

In one example, the emotional response engine is a morality engine, the predicted emotional state set is a predicted human morality vector, the determined emotional state sets are determined human morality vectors, and the processor(s) is configured for updating the morality engine based on the predicted human morality vector and the determined human morality vectors. The processor(s) may be configured for deriving a single reference human morality vector from the determined human morality vectors, comparing the single reference human morality vector and the predicted human morality vector, generating at least one error based on the comparison, and updating the morality engine based on the error(s).

In another example, the emotional response engine is a kindness engine, the predicted emotional state set is a predicted kindness level, the determined emotional state sets are determined kindness levels, and the processor(s) is configured for updating the kindness engine based on the predicted kindness level and the determined kindness levels. The processor(s) may be further configured for deriving a single reference human kindness level from the determined kindness levels, comparing the single reference human kindness level and the predicted kindness level, generating at least one error based on the comparison, and updating the kindness engine based on the error(s).

In accordance with a second aspect of the present inventions, a method of training an emotional response engine for use in an artificial intelligence (AI) system is provided. The method comprises determining a range of use of the AI control system, inputting a real-life scenario that may occur in the context of the AI control system into an emotional response engine, outputting a predicted emotional state set from the emotional response engine in response to the input of the real-life scenario into the emotional response engine, presenting the potential action outcome to each of a plurality of human subjects, and detecting brain activity of the human subjects in response to presenting the potential action outcome to each of the human subjects.

The method further comprises determining a plurality of emotional state sets respectively for the human subjects based on the detected brain activity of the human subject, and updating the emotional response engine based on the predicted emotional state set and the determined emotional state sets. One method further comprises reducing the determined emotional state sets into a single reference emotional state set representative of a collective emotional response of the human subjects, comparing the single reference emotional state set and the predicted emotional state set, and generating at least one error based on the comparison. In this case, the emotional response engine is updated based on the error(s).

In one example, the emotional response engine is a morality engine, the predicted emotional state set is a predicted human morality vector, the determined emotional state sets are determined human morality vectors, and the morality engine is updated based on the predicted human morality vector and the determined emotional states. This method may further comprise deriving a single reference human morality vector from the determined human morality vectors, comparing the single reference human morality vector and the predicted human morality vector, generating at least one error based on the comparison, and updating the morality engine based on the error(s).

In another example, the emotional response engine is a kindness engine, the predicted emotional state set is a predicted kindness level, the determined emotional state sets are determined kindness levels, and the kindness engine is updated based on the predicted kindness level and the determined kindness levels. This method may further comprise deriving a single reference human kindness level from the determined kindness levels, comparing the single reference human kindness level and the predicted kindness level, generating at least one error based on the comparison, and updating the kindness engine based on the error(s).

In accordance with a third aspect of the present inventions, an artificial intelligence (AI) control system is provided. The AI control system comprises memory configured for storing an emotional response engine, and at least one sensor configured for sensing an external environment of the artificial intelligence system. The AI control system further comprises at least one processor is configured for generating a plurality of real-life scenarios based on the sensed external environment, inputting each of the real-life scenarios into the emotional response engine, such that the emotional response engine respectively outputs a plurality of predicted emotional state sets. In one example, the emotional response engine is a morality engine, and the predicted emotional state sets are predicted human emotional response vectors. In another example, the emotional response engine is a kindness engine, and the predicted emotional state sets are predicted human kindness levels.

The processor(s) is further configured for inputting the predicted emotional state sets into a cost function or a reward function (which may comprise probabilities of outcomes associated with the real-life scenarios), such that the cost function or reward function outputs a plurality of scores respectively for the real-life scenarios, selecting one of the real-life scenarios based on the scores (e.g., the real-life scenario corresponding to the best score), and one or more actuators configured for performing an action associated with the selected real-life scenario (e.g., at least one of modifying a speed of a vehicle and changing a direction of the vehicle). In one embodiment, the processor(s) is configured for determining a level of performance of a primary objective of the AI control system, in which case, the cost function or reward function may comprise a weighting dependent on the determined performance level of the primary objective of the AI control system.

In accordance with a fourth aspect of the present inventions, a method of operating an artificial intelligence (AI) control system is provided. The method comprises sensing an external environment, generating a plurality of real-life scenarios based on the sensed external environment, inputting each of the real-life scenarios into an emotional response engine, and outputting a plurality of predicted emotional state sets from the emotional response engine. In one example, the emotional response engine is a morality engine, and the predicted emotional state sets are predicted human emotional response vectors. In another example, the emotional response engine is a kindness engine, and the predicted emotional state sets are predicted human kindness levels.

The method further comprises inputting the predicted emotional state sets into a cost function or a reward function (which may comprise probabilities of outcomes associated with the real-life scenarios), outputting a plurality of scores from the cost function or reward function respectively for the real-life scenarios, selecting one of the real-life scenarios based on the scores (e.g., the real-life scenario corresponding to the best score), and performing an action associated with the selected real-life scenario (e.g., at least one of modifying a speed of a vehicle and changing a direction of the vehicle). One method further comprises determining a level of performance of a primary objective of the AI control system, in which case, the cost function or reward function may comprise a weighting dependent on the determined performance level of the primary objective of the AI control system.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a block diagram of one embodiment of an emotional response engine generation system constructed in accordance with the present inventions;

FIG. 2 is a flow diagram illustrating one method of using the emotional response engine generation system to generate reference emotional state sets from a group of human subjects;

FIG. 3 is a flow diagram illustrating one method of using the emotional response engine generation system to train an emotional response engine;

FIG. 4 is a block diagram of an artificial intelligence (AI) control system that utilizes the trained emotional response engine;

FIG. 5 is a severity-probability matrix utilized by the AI control system of FIG. 4; and

FIG. 6 is a flow diagram illustrating one method of using the AI control system of FIG. 4 to make a decision.

### DETAILED DESCRIPTION OF THE EMBODIMENTS

The present disclosure is directed to an emotional response engine, which is created based on brain activity measured form one or more humans while observing the outcome of possible events (actions). Once developed, the emotional response engine can be deployed into any artificial intelligence (AI) control system as part of a cost-function or reward-function where there is a need to know how a human observer would interpret certain outcomes (actions).

The general process of developing the emotional response engine includes generating a large dataset of human brain activity related to observing the outcome of actions. Some of these actions will have positive outcomes and some will have negative outcomes. The measured brain activity could be related to joy, excitement, relaxation, surprise, fear, stress, anxiety, sadness, anger, disgust, contempt, contentment, calmness, approval, etc., through a secondary translator algorithm that predicts the resulting emotional state(s). The general process of developing the emotional response engine also includes training the emotional response engine to predict the human brain activity and the resulting emotional state(s) associated with an outcome. The emotional response engine may then be used to provide scoring information to an AI control system that has to make a decision to optimize human emotions related to an action.

Significantly, the present disclosure is not concerned about recording the morality of conscious human decision making and using the information gleaned from the recorded morality to provide moral guidance to AI control systems, but rather is concerned about recording the visceral neuronal and emotional response of a moral person observing actions and resulting outcomes and then subsequently predicting those neuronal and emotional responses to guide the decision making of AI control systems.

It is noted that recording the conscious human decision-making process may not always be indicative of the sentiments of any particular person with regard to the morality of certain actions. For example, some people may not be truthful in their responses to questions of morality for fear of being judged or may tend to consciously respond to questions of morality how they think the person who formulated the questions wants them to respond. Furthermore, it may be difficult for some people to understand their own primary or secondary emotional response to questions of morality, so that they may not be capable of truthfully conveying their emotional response to questions of morality.

In the illustrated embodiments, the emotional response engine takes the form of a “neurome” that can be created, trained, and then used in an AI control system. Generically, a “neurome” can be defined as a component in which stimuli (e.g., video, audio, text, etc.) from one or more sources of content (e.g., a movie, a book, a song, a household appliance, an automobile, food, artwork, or sources of consumable chemical substances (where the chemical substances can include, e.g., caffeinated drinks, soft drinks, energy drinks, tobacco products, drugs (pharmaceutical or recreational), etc.) can be input and from which a brain state, predictive of the brain state of the user or from which a predicted brain state, behavior, preferences, or attitude of the user, can be derived, is output as if the user received the same stimuli. The present disclosure utilizes the neurome to predict one or more emotional states of one or more people in response to an input of a moral dilemma to the neurome, which predicted emotional state(s) are then utilized by the AI to make a decision. Further details discussing the creation, training, and use of neuromes can be found in U.S. Provisional Application 63/047,991, entitled “Systems and Methods for Training and Using a Neurome that Emulates the Brain of a User,” which is expressly incorporated herein by reference.

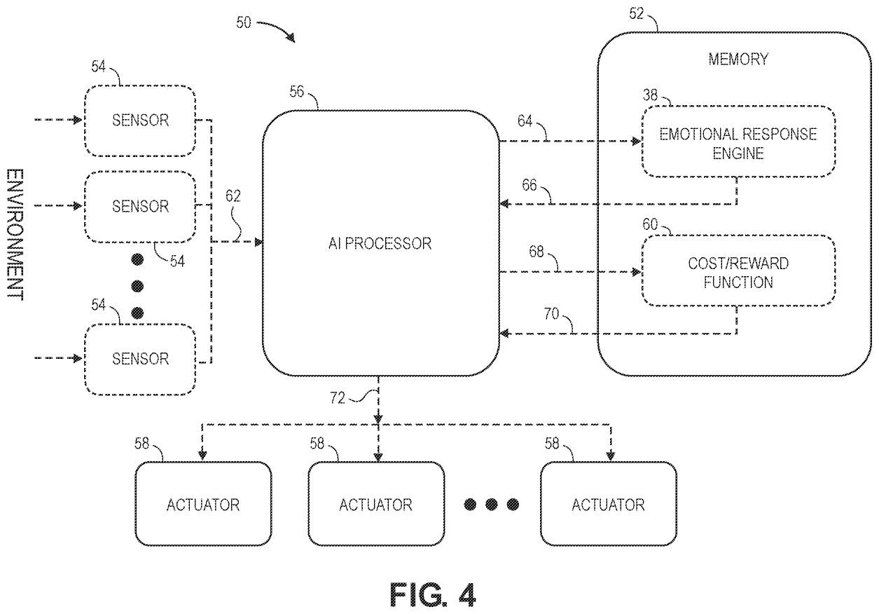

In one non-limiting embodiment described below, the emotional response engine takes the form of morality engine (ME), which provides oversight for the AI control system 50 (shown in FIG. 4) to reduce nonhuman or inhumane behaviors and actions, and can be incorporated into any AI control system 50 that has the potential to cause injury to people, property, animals, or the environment, and allows the AI control system 50 to select actions that are in synch with a particular cultural morality. The ME can be modeled from a single person, but preferably is modeled from a large population pool of human subjects from either a single culture or multiple cultures.

In another non-limiting embodiment further described below, the emotional response engine takes the form of a kindness engine (KE). If the AI control system 50 has developed unethical strategies, the KE might predict that a human observer would interpret these actions as being unkind and thus be less likely to choose them. The KE can be modeled from a single person, but preferably is modeled from a large population pool of human subjects from either a single culture or multiple cultures. However, unlike morality, which is culturally variable and could be heavily biased, kindness is a more basic response or action that can be easily identified by both humans and animals.

It should be appreciated that although the emotional response engines, e.g., ME and KE, described herein lend themselves well to providing oversight to the AI control system 50 in terms of morality or kindness, emotional response engines can also be used to provide oversight to the AI control system 50 in any context where it is desired to provide human emotional feedback to any AI control system, such that the AI control system may select appropriate actions that are in accordance with a desired human emotional response to that selected action.

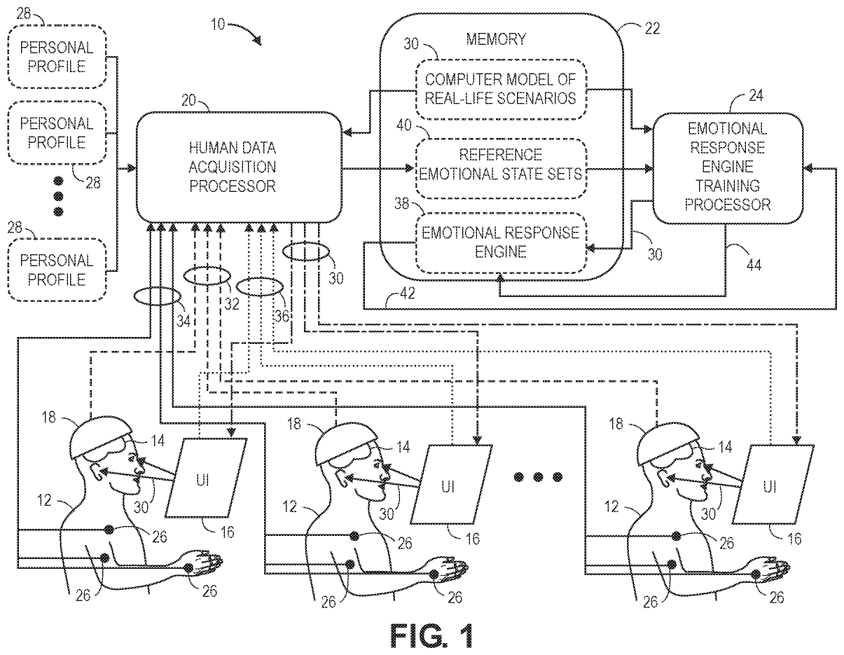

Referring now to FIG. 1, one embodiment of an emotional response engine generation system 10 will be described. The emotional response engine generation system 10 generally comprises one or more user interfaces (UI) 16 (in this case, a plurality of UIs), one or more non-invasive brain interface assemblies 18 (in this case, a plurality of non-invasive brain interface assemblies), a human data acquisition processor 20, memory 22, and an emotional response engine training processor 24. The emotional response engine generation system 10 optionally comprises a plurality of sets of peripheral sensors 26 and a plurality of on-line personal profiles 28 (e.g., one or more of an internet browsing history of the human subjects 12, a reading history of the human subjects 12, and autobiographical information of the human subjects 12).

The human data acquisition processor 20 is configured for generating and storing a computer model of real-life scenarios 30, and subsequently presenting the computer model of real-life scenarios 30 to each of the plurality of human subjects 12 via the respective UIs 16 (via, e.g., a display and/or speaker). The computer model of real-life scenarios 30 may comprise a list of actions and potential outcomes that may occur in the context of a range of use of an AI control system (e.g., operating or controlling autonomous cars). For example, the actions may demonstrate morality (or no morality) in the case where the emotional response engine to be trained takes the form of an ME. As another example, the actions may demonstrate kindness (or no kindness) in the case where the emotional response engine to be trained takes the form of a KE) in the memory 22.

In one embodiment, the potential outcomes may take the form of text, e.g., “The car strikes and kills a dog but does not injure a child” in the case where the emotional response engine takes the form of an ME, or “Traffic is not stopped to allow a mother duck and her ducklings to cross the road” in the case where the emotional response engine takes the form of a KE. In another embodiment, the potential outcomes may take the form of virtual audio/video/tactile simulations of the actions (e.g., real-life scenarios), e.g., a video showing a car striking and killing a dog, but not injuring a child, in the case where the emotional response engine takes the form of an ME, or a video showing traffic not stopping to allow a mother duck and her ducklings to cross the road, in the case where the emotional response engine takes the form of an ME. As computer simulation tools improve in terms of power and efficiency, the quality of the audio/video/tactile simulations will improve. The potential outcomes may be simulated from the perspective of a passive participant or an active participant.

Each non-invasive brain interface assembly 18 is configured for detecting neural activity 32 in the brain 14 (i.e., the brain activity) of the human subject 12 in response to the presentation of the computer model of real-life scenarios 30 to the respective human subject 12. Each non-invasive brain interface assembly 18 may be any device capable of non-invasively acquiring hi-fidelity signals representing the brain activity 32 in the brain 14 of the human subject 12. In the preferred embodiment, each non-invasive brain interface assembly 18 is portable and wearable. Although multiple brain interface assemblies 18 are illustrated for use by multiple human subjects 12 (e.g., in a parallel simultaneous manner), it should be appreciated that a single brain interface assembly 18 can be used by multiple human subjects 12 (e.g., in a serial manner). Any one of variety of embodiments of brain interface assemblies 18 may be used in the emotional response engine generation system 10.

In one embodiment, each non-invasive brain interface assembly 18 may be an optically-based non-invasive brain interface assembly. For example, each non-invasive brain interface assembly 18 may, e.g., incorporate any one or more of the brain activity detection technologies described in U.S. patent application Ser. No. 15/844,370, entitled “Pulsed Ultrasound Modulated Optical Tomography Using Lock-In Camera” (now U.S. Pat. No. 10,335,036), U.S. patent application Ser. No. 15/844,398, entitled “Pulsed Ultrasound Modulated Optical Tomography With Increased Optical/Ultrasound Pulse Ratio” (now U.S. Pat. No. 10,299,682), U.S. patent application Ser. No. 15/844,411, entitled “Optical Detection System For Determining Neural Activity in Brain Based on Water Concentration” (now U.S. Pat. No. 10,420,469), U.S. patent application Ser. No. 15/853,209, entitled “System and Method For Simultaneously Detecting Phase Modulated Optical Signals” (now U.S. Pat. No. 10,016,137), U.S. patent application Ser. No. 15/853,538, entitled “Systems and Methods For Quasi-Ballistic Photon Optical Coherence Tomography In Diffusive Scattering Media Using a Lock-In Camera” (now U.S. Pat. No. 10,219,700), U.S. patent application Ser. No. 16/266,818, entitled “Ultrasound Modulating Optical Tomography Using Reduced Laser Pulse Duration,” U.S. patent application Ser. No. 16/299,067, entitled “Non-Invasive Optical Detection Systems and Methods in Highly Scattering Medium,” U.S. patent application Ser. No. 16/379,090, entitled “Non-Invasive Frequency Domain Optical Spectroscopy For Neural Decoding,” U.S. patent application Ser. No. 16/382,461, entitled “Non-Invasive Optical Detection System and Method,” U.S. patent application Ser. No. 16/392,963, entitled “Interferometric Frequency-Swept Source And Detector In A Photonic Integrated Circuit,” U.S. patent application Ser. No. 16/392,973, entitled “Non-Invasive Measurement System and Method Using Single-Shot Spectral-Domain Interferometric Near-Infrared Spectroscopy Based On Orthogonal Dispersion, U.S. patent application Ser. No. 16/393,002, entitled “Non-Invasive Optical Detection System and Method Of Multiple-Scattered Light With Swept Source Illumination,” U.S. patent application Ser. No. 16/385,265, entitled “Non-Invasive Optical Measurement System and Method for Neural Decoding,” U.S. patent application Ser. No. 16/533,133, entitled “Time-Of-Flight Optical Measurement And Decoding Of Fast-Optical Signals,” U.S. patent application Ser. No. 16/565,326, entitled “Detection Of Fast-Neural Signal Using Depth-Resolved Spectroscopy,” U.S. patent application Ser. No. 16/226,625, entitled “Spatial and Temporal-Based Diffusive Correlation Spectroscopy Systems and Methods,” U.S. Provisional Application Ser. No. 62/772,584, entitled “Diffuse Correlation Spectroscopy Measurement Systems and Methods,” U.S. patent application Ser. No. 16/432,793, entitled “Non-Invasive Measurement Systems with Single-Photon Counting Camera,” U.S. patent application Ser. No. 16/842,443, entitled “Interferometric Parallel Detection Using Digital Rectification and Integration”, U.S. patent application Ser. No. 16/842,488, entitled “Interferometric Parallel Detection Using Analog Data Compression,” U.S. patent application Ser. No. 16/842,523, entitled “Partially Balanced Interferometric Parallel Detection,” which are all expressly incorporated herein by reference in their entirety.

In another embodiment, each non-invasive brain interface assembly 18 may be an optically-based, time-domain, non-invasive brain interface assembly. For example, each non-invasive brain interface assembly 18 may, e.g., incorporate any one or more of the brain activity detection technologies described in U.S. Non-Provisional application Ser. No. 16/051,462, entitled “Fast-Gated Photodetector Architecture Comprising Dual Voltage Sources with a Switch Configuration” (now U.S. Pat. No. 10,158,038), U.S. patent application Ser. No. 16/202,771, entitled “Non-Invasive Wearable Brain Interface Systems Including a Headgear and a Plurality of Self-Contained Photodetector Units Configured to Removably Attach to the Headgear” (now U.S. Pat. No. 10,340,408), U.S. patent application Ser. No. 16/283,730, entitled “Stacked Photodetector Assemblies” (now U.S. Pat. No. 10,515,993), U.S. patent application Ser. No. 16/544,850, entitled “Wearable Systems with Stacked Photodetector Assemblies” (now U.S. Pat. No. 10,847,563), U.S. patent application Ser. No. 16/844,860, entitled “Photodetector Architectures for Time-Correlated Single Photon Counting,” U.S. patent application Ser. No. 16/852,183, entitled “Photodetector Architectures for Efficient Fast-Gating,” U.S. patent application Ser. No. 16/880,686, entitled “Photodetector Systems with Low-Power Time-To-Digital Converter Architectures” (now U.S. Pat. No. 10,868,207), U.S. Provisional Application Ser. No. 62/979,866, entitled “Optical Module Assemblies,” U.S. Provisional Application Ser. No. 63/038,485, entitled “Control Circuit for a Light Source in an Optical Measurement System,” U.S. Provisional Application Ser. No. 63/040,773, entitled “Multiplexing Techniques for Interference Reduction in Time-Correlated Signal Photon Counting,” U.S. Provisional Application Ser. No. 63/064,249, entitled “Maintaining Consistent Photodetector Sensitivity in an Optical Measurement System,” U.S. Provisional Application Ser. No. 63/027,018, entitled “Phase Lock Loop Circuit Based Adjustment of a Measurement Time Window in an Optical Measurement System,” U.S. Provisional Application Ser. No. 63/044,521, entitled “Techniques for Determining a Timing Uncertainty of a Component of an Optical Measurement System,” U.S. Provisional Application Ser. No. 63/059,382, entitled “Techniques for Characterizing a Nonlinearity of a Time-To-Digital Converter in an Optical Measurement System,” U.S. Provisional Application Ser. No. 63/027,025, entitled “Temporal Resolution Control for Temporal Point Spread Function Generation in an Optical Measurement System,” U.S. Provisional Application Ser. No. 63/057,080, entitled “Bias Voltage Generation in an Optical Measurement System,” U.S. Provisional Application Ser. No. 63/051,099, entitled “Detection of Motion Artifacts in Signals Output By Detectors of a Wearable Optical Measurement System,” U.S. Provisional Application Ser. No. 63/057,077, entitled “Dynamic Range Optimization in an Optical Measurement System,” U.S. Provisional Application Ser. No. 63/074,721, entitled “Maintaining Consistent Photodetector Sensitivity in an Optical Measurement System,” U.S. Provisional Application Ser. No. 63/070,123, entitled “Photodetector Calibration of an Optical Measurement System,” U.S. Provisional Application Ser. No. 63/071,473, entitled “Estimation of Source-Detector Separation in an Optical Measurement System,” U.S. Provisional Application Ser. No. 63/081,754, entitled “Wearable Module Assemblies for an Optical Measurement System,” U.S. Provisional Application Ser. No. 63/086,350, entitled “Wearable Devices and Wearable Assemblies with Adjustable Positioning for Use in an Optical Measurement System,” U.S. Provisional Application Ser. No. 63/038,459, entitled “Integrated Detector Assemblies for a Wearable Module of an Optical Measurement System,” U.S. Provisional Application Ser. No. 63/038,468, entitled “Detector Assemblies for a Wearable Module of an Optical Measurement System and Including Spring-Loaded Light-Receiving Members,” U.S. Provisional Application Ser. No. 63/038,481, entitled “Integrated Light Source Assembly with Laser Coupling for a Wearable Optical Measurement System,” U.S. Provisional Application Ser. No. 63/079,194, entitled “Multimodal Wearable Measurement Systems and Methods,” U.S. Provisional Application Ser. No. 63/064,688, entitled “Time Domain-Based Optical Measurement System and Method Configured to Measure Absolute Properties of Tissue,” and U.S. Provisional Application Ser. No. 63/120,650, entitled “Systems, Circuits, and Methods for Reducing Common-Mode Noise in Biopotential Recordings,” which are all expressly incorporated herein by reference.

The optically-based, time-domain, non-invasive brain interface assembly may also include a wearable modular assembly (e.g., non-invasive brain interface assembly 18) configured to be worn on the head of a user (e.g., human subject 12) and wherein the wearable modular assembly includes a plurality of connectable wearable modules. Each wearable module includes a light source configured to emit a light pulse toward a target within the brain 14 of the human subject 12 and a plurality of detectors configured to receive photons included in the light pulse after the photons are scattered by the target. The wearable module assemblies can conform to a 3D surface of the human subject's head, maintain tight contact of the detectors with the human subject's head to prevent detection of ambient light, and maintain uniform and fixed spacing between light sources and detectors. The wearable module assemblies may also accommodate a large variety of head sizes, from a young child's head size to an adult head size, and may accommodate a variety of head shapes and underlying cortical morphologies through the conformability and scalability of the wearable module assemblies. These exemplary modular assemblies and systems are described in more detail in U.S. Provisional Applications Nos. 63/038,459, 63/038,468, 63/038,481, 63/064,688, 63/081,754, and 63/086,350, which applications have been previously incorporated herein by reference in their respective entireties.

Example time domain-based optical measurement techniques include, but are not limited to, time-correlated single-photon counting (TCSPC), time domain near infrared spectroscopy (TD-NIRS), time domain diffusive correlation spectroscopy (TD-DCS), and time domain digital optical tomography (TD-DOT).

In still another embodiment, each non-invasive brain interface assembly 18 may be a magnetically-based non-invasive brain interface assembly. For example, each non-invasive brain interface assembly 18 may, e.g., incorporate any one or more of the brain activity detection technologies described in U.S. patent application Ser. No. 16,428,871, entitled “Magnetic Field Measurement Systems and Methods of Making and Using,” U.S. patent application Ser. No. 16/418,478, entitled “Magnetic Field Measurement System and Method of Using Variable Dynamic Range Optical Magnetometers”, U.S. patent application Ser. No. 16/418,500, entitled, “Integrated Gas Cell and Optical Components for Atomic Magnetometry and Methods for Making and Using,” U.S. patent application Ser. No. 16/457,655, entitled “Magnetic Field Shaping Components for Magnetic Field Measurement Systems and Methods for Making and Using,” U.S. patent application Ser. No. 16/213,980, entitled “Systems and Methods Including Multi-Mode Operation of Optically Pumped Magnetometer(S),” (now U.S. Pat. No. 10,627,460), U.S. patent application Ser. No. 16/456,975, entitled “Dynamic Magnetic Shielding and Beamforming Using Ferrofluid for Compact Magnetoencephalography (MEG),” U.S. patent application Ser. No. 16/752,393, entitled “Neural Feedback Loop Filters for Enhanced Dynamic Range Magnetoencephalography (MEG) Systems and Methods,” U.S. patent application Ser. No. 16/741,593, entitled “Magnetic Field Measurement System with Amplitude-Selective Magnetic Shield,” U.S. patent application Ser. No. 16/820,131, entitled “Integrated Magnetometer Arrays for Magnetoencephalography (MEG) Detection Systems and Methods,” U.S. patent application Ser. No. 16/850,380, entitled “Systems and Methods for Suppression of Interferences in Magnetoencephalography (MEG) and Other Magnetometer Measurements,” U.S. patent application Ser. No. 16/850,444, entitled “Compact Optically Pumped Magnetometers with Pump and Probe Configuration and Systems and Methods,” U.S. Provisional Application Ser. No. 62/842,818, entitled “Active Shield Arrays for Magnetoencephalography (MEG),” U.S. patent application Ser. No. 16/928,810, entitled “Systems and Methods for Frequency and Wide-Band Tagging of Magnetoencephalography (Meg) Signals,” U.S. patent application Ser. No. 16/984,720, entitled “Systems and Methods for Multiplexed or Interleaved Operation of Magnetometers,” U.S. patent application Ser. No. 16/984,752, entitled “Systems and Methods having an Optical Magnetometer Array with Beam Splitters,” U.S. patent application Ser. No. 17/004,507, entitled “Methods and Systems for Fast Field Zeroing for Magnetoencephalography (MEG),” U.S. patent application Ser. No. 16/862,826, entitled “Single Controller for Wearable Sensor Unit that Includes an Array Of Magnetometers,” U.S. patent application Ser. No. 16/862,856, entitled “Systems and Methods for Measuring Current Output By a Photodetector of a Wearable Sensor Unit that Includes One or More Magnetometers,” U.S. patent application Ser. No. 16/862,879, entitled “Interface Configurations for a Wearable Sensor Unit that Includes One or More Magnetometers,” U.S. patent application Ser. No. 16/862,901, entitled “Systems and Methods for Concentrating Alkali Metal Within a Vapor Cell of a Magnetometer Away from a Transit Path of Light,” U.S. patent application Ser. No. 16/862,919, entitled “Magnetic Field Generator for a Magnetic Field Measurement System,” U.S. patent application Ser. No. 16/862,946, entitled “Magnetic Field Generator for a Magnetic Field Measurement System,” U.S. patent application Ser. No. 16/862,973, entitled “Magnetic Field Measurement Systems Including a Plurality of Wearable Sensor Units Having a Magnetic Field Generator,” U.S. Provisional Application Ser. No. 63/035,629, entitled “Self-Calibration of Flux Gate Offset and Gain Drift To Improve Measurement Accuracy of Magnetic Fields from the Brain Using a Wearable Neural Detection System,” U.S. Provisional Application Ser. No. 63/035,650, entitled “Nested and Parallel Feedback Control Loops for Ultra-Fine Measurements of Magnetic Fields from the Brain Using a Neural Detection System,” U.S. Provisional Application Ser. No. 63/035,664, entitled “Estimating the Magnetic Field at Distances from Direct Measurements to Enable Fine Sensors to Measure the Magnetic Field from the Brain Using a Neural Detection System,” U.S. Provisional Application Ser. No. 63/035,683, entitled “Systems and Methods that Exploit Maxwell's Equations and Geometry to Reduce Noise for Ultra-Fine Measurements of Magnetic Fields From the Brain Using a Neural Detection System,” U.S. Provisional Application Ser. No. 63/035,680, entitled “Optimal Methods to Feedback Control and Estimate Magnetic Fields to Enable a Neural Detection System to Measure Magnetic Fields from the Brain,” U.S. Provisional Application Ser. No. 62/983,406, entitled “Two Level Magnetic Shielding of Magnetometers,” U.S. Provisional Application Ser. No. 63/076,015, entitled “Systems and Methods for Recording Neural Activity,” U.S. Provisional Application Ser. No. 63/058,616, entitled “OPM Module Assembly with Alignment and Mounting Components as Used in a Variety of Headgear Arrangements,” U.S. Provisional Application Ser. No. 63/076,880, entitled “Systems and Methods for Multimodal Pose and Motion Tracking for Magnetic Field Measurement or Recording Systems,” and U.S. Provisional Application Ser. No. 63/080,248, entitled “Optically Pumped Magnetoencephalography (OP-MEG) Validation with Optical Tracking Data,” which are all expressly incorporated herein by reference in their entirety. The magnetically-based non-invasive brain interface assembly can be used in a magnetically shielded environment as described for example in U.S. Provisional Application Ser. No. 63/076,015, entitled “Systems and Methods for Recording Neural Activity,” which is expressly incorporated herein by reference in its entirety.

The magnetically-based non-invasive brain interface assembly may also include a plurality of optically pumped magnetometer (OPM) modular assemblies, which OPM modular assemblies are enclosed within a housing sized to fit into a headgear (e.g., non-invasive brain interface assembly 18) for placement on a head of a user (e.g., human subject 12). The OPM modular assembly is designed to enclose the elements of the OPM optics, vapor cell, and detectors in a compact arrangement that can be positioned close to the head of the human subject 12. The headgear may include an adjustment mechanism used for adjusting the headgear to conform with the human subject's head. These exemplary OPM modular assemblies and systems are described in more detail in U.S. Provisional Application No. 63/058,616, previously incorporated by reference in its entirety.

Example techniques of using the magnetically-based non-invasive brain interface assembly are directed to the area of magnetic field measurement systems including systems for magnetoencephalography (MEG).

The sets of peripheral sensors 26 are configured for, in response to the presentation of the computer model of real-life scenarios 30 to the respective human subjects 12, detecting peripheral physiological functions 34 of the human subjects 12, e.g., heart rate, respiratory rate, blood pressure, skin conductivity, etc. The UIs 16 may optionally be configured for, in response to the presentation of the computer model of real-life scenarios 30 to the respective human subjects 12, receiving conscious input 36 (via, e.g., a keyboard, microphone, button, remote control, etc.) from the human subjects 12 indicating emotional states of the human subjects 12. For example, as the computer model of real-life scenarios 30 are presented to the human subjects 12, the human subjects 12 can be queried to provide the conscious input 36 via the respective UIs 16 indicating the emotional states perceived by the user 12. The query can either be opened ended, multiple choice, or binary (i.e., yes or no).

The human data acquisition processor 20 is further configured for determining a plurality of emotional state sets respectively for the human subjects 12 based on the detected brain activity 32 of the human subjects 12 for each real-life scenario of the computer model 30 (i.e., for each real-life scenario 30, a plurality of emotional states sets respectively corresponding to human subjects 12 will be generated). Each emotional state set may contain one or more emotional states (e.g., joy, excitement, relaxation, surprise, fear, stress, anxiety, sadness, anger, disgust, contempt, contentment, calmness, approval, etc.).

The emotional state(s) of each human subject 12 may be determined based on the detected brain activity 32 in any one of a variety of manners. In one embodiment, a univariate approach may be performed in determining the emotional state(s) of the human subject 12, i.e., the brain activity 32 can be detected in a plurality (e.g., thousands) of separable cortical modules of the human subject 12, and the brain activity 32 obtained from each cortical module can be analyzed separately and independently. In another embodiment, a multivariate approach may be performed in determining the emotional state(s) of the human subject 12, i.e., the brain activity 32 can be detected in a plurality (e.g., thousands) of separable cortical modules of the human subject 12, and the full spatial pattern of the brain activity 32 obtained from the cortical modules can be assessed together.

A variety of models may be used to classify the emotional state(s) of the human subject 12, which will highly depend on the characteristics of brain activity 32 that are input into the models. Selection of the characteristics of brain activity 32 to be input into the models must be considered in reference to univariate and multivariate approaches, since the univariate approach, e.g., focuses on a single location, and therefore will not take advantage of features that correlate multiple locations. Selecting a model will be heavily dependent on whether the data is labeled or unlabeled (meaning is it known what the human subject 12 is doing at the time that the brain activity 32 is detected), as well as many other factors (e.g., is the data assumed to be normally distributed, is the data assumed relationship linear, is the data assumed relationship non-linear, etc.) Models can include, e.g., support vector machines, expectation maximization techniques, naïve-Bayesian techniques, neural networks, simple statistics (e.g., correlations), deep learning models, pattern classifiers, etc.

These models are typically initialized with some training data (meaning that a calibration routine can be performed on the human subject 12 to determine what the human subject 12 is doing). If no training information can be acquired, such models can be heuristically initialized based on prior knowledge, and the models can be iteratively optimized with the expectation that optimization will settle to some optimal maximum or minimum solution. Once it is known what the human subject 12 is doing, the proper characteristics of the brain activity 32 and proper models can be queried. The models may be layered or staged, so that, e.g., a first model focuses on pre-processing data (e.g., filtering), the next model focuses on clustering the pre-processed data to separate certain features that may be recognized to correlate with a known activity performed by the human subject 12, and then the next model can query a separate model to determine the emotional state(s) based on that human subject activity.

Training data or prior knowledge of the human subject 12 may be obtained by providing known life/work context to the human subject 12. Altogether, the models can be used to track the emotional state(s) and perception under natural or quasi-natural (i.e., in response to providing known life/work context to the user) and dynamic conditions taking in the time-course of averaged activity and determining the brain state of the user based on constant or spontaneous fluctuations in the characteristics of the brain activity 32 extracted from the data.

A set of data models that have already been proven, for example in a laboratory setting, can be initially uploaded, which can then be used to determine the emotional state(s) of the human subject 12. Optionally, data can be collected during actual use with the human subject 12, which can then be downloaded and analyzed in a separate server, for example in a laboratory setting, to create new or updated models. Software upgrades, which may include the new or updated models, can be uploaded to provide new or updated data modelling and data collection.

Further details regarding determining the emotional state(s) of a person based on detected brain activity 32 can be found in a variety of peer-reviewed publications. See, e.g., Lee, B. T., Seok, J. H., Lee., B. C, Cho, S. W., Chai, J. H., Choi, I. G., Ham, B. J., “Neural correlates of affective processing in response to sad and angry facial stimuli in patients with major depressive disorder,” Prog Neuropsychopharmacol Biol Psychiatry, 32(3), 778-85 (2008); A. C. Felix-Ortiz, A. C., Burgos-Robles, A., Bhagat, N. D., Leppla, C. A., Tye, K. M., “Bidirectional modulation of anxiety-related and social behaviors by amygdala projections to the medial prefrontal cortex,” Neuroscience 321, 197-209 (2016); Beauregard, M., Levesque, J. & Bourgouin, P., “Neural correlates of conscious self-regulation of emotion,” J. Neurosci. (2001): 21, RC165; Phan, K. L., Wager, T., Taylor, S. F. & Liberzon, I., “Functional neuroanatomy of emotion: a meta-analysis of emotion activation studies in PET and fMRI,” Neuroimage, 16, 331-348 (2002); Canli, T. & Amin, Z., “Neuroimaging of emotion and personality: scientific evidence and ethical considerations,” Brain Cogn., 50, 414-431 (2002), McCloskey, M. S., Phan, K. L. & Coccaro, E. F., “Neuroimaging and personality disorders,” Curr. Psychiatry Rep., 7, 65-72 (2005); Heekeren, H. R., Marrett, S., Bandettini, P. A. & Ungerleider, L. G., “A general mechanism for perceptual decision-making in the human brain,” Nature, 431, 859-862 (2004); Shin L M, Rauch S L, Pitman R K. Amygdala, Medial Prefrontal Cortex, and Hippocampal Function in PTSD, Ann N Y Acad Sci., 1071(1) (2006); Lis E, Greenfield B, Henry M, Guile J M, Dougherty G., “Neuroimaging and genetics of borderline personality disorder: a review,” J Psychiatry Neurosci., 32(3), 162-173 (2007); Etkin A, Wager T D, “Functional neuroimaging of anxiety: a meta-analysis of emotional processing in PTSD, social anxiety disorder, and specific phobia,” Am J Psychiatry, 164(10), 1476-1488 (2007); Etkin A. Functional Neuroimaging of Major Depressive Disorder: A Meta-Analysis and New Integration of Baseline Activation and Neural Response Data, Am J Psychiatry, 169(7), 693-703 (2012); Sheline Y I, Price J L, Yan Z, Mintun M A, “Resting-state functional MRI in depression unmasks increased connectivity between networks via the dorsal nexus, Proc Natl Acad Sci., 107(24), 11020-11025 (2010); Bari A, Robbins T W, “Inhibition and impulsivity: Behavioral and neural basis of response control,” Prog Neurobiol., 108:44-79 (2013); Kagias, Konstantinos et al. “Neuronal responses to physiological stress,” Frontiers in genetics, 3:222 (2012).

The human data acquisition processor 20 may be configured for determining the emotional state sets respectively for the human subjects 12 further based on peripheral physiological functions 34 detected by the peripheral sensors 26 in response to the presentation of each real-life scenario 30 of the computer model to the respective human subjects 12. That is, the peripheral physiological functions 34 of the human subjects 12, e.g., heart rate, respiratory rate, blood pressure, skin conductivity, etc., may inform the emotional states of the human subjects 12 that have been determined in response to the presentation of the computer model of real-life scenarios 30 to the respective human subjects 12. The human data acquisition processor 20 may be configured for determining the emotional state sets respectively for the human subjects 12 further based on the conscious input 36 received by the human subjects 12 via the UIs 16 and/or the personal profiles 28 in response to the presentation of the computer model of real-life scenarios 30 to the respective human subjects 12.

In the case where the emotional response engine to be trained is an ME, the emotional state sets may take the form of human morality vectors. Each human emotional response vector may contain a weighted or unweighted value correlated to the strength of a particular emotional state (e.g., from −1 to +1 or from 0 to +1), which may be summed to create a morality score indicative of a positive or negative emotional response of the human subject to the action. In one embodiment, the values of the human emotional response vector associated with the emotional states that are indicative of negative emotional responses to the action are assigned to be positive or high (the more negative the emotional response the more positive or high the value), and the values of the human emotional response vector associated with the emotional states that are indicative of positive emotional responses to the action are assigned to be negative or lower (the more positive the emotional response the more negative or lower the value). Of course, the manner in which the values of the human emotional response vector is assigned to the emotional states are arbitrary, and any value assignment technique that results in a human emotional response vector indicative of the emotional response of the human subject to the action can be used.

In the case where the emotional response engine to be trained is a KE, the emotional state sets may take the form of human kindness levels. Each human kindness level may contain a weighted or unweighted value correlated to the strength of kindness level (e.g., from −1 to +1 or from 0 to +1). In one embodiment, the human kindness levels indicative of kindness to the action are assigned to be positive or high (the more kindness the more positive or high the value), and the human kindness levels indicative of no kindness to the action are assigned to be negative or lower (the less kindness the more negative or lower the value). Of course, the manner in which the human kindness levels are assigned to the kindness levels are arbitrary, and any value assignment technique that results in a human kindness level indicative of the kindness of the human subject to the action may be used.

The human data acquisition processor 20 is further configured for reducing the emotional state sets determined for the human subjects 12 into a single reference emotional state set 40 representative of the collective emotional response of the human subjects 12 for each real-life scenario of the computer model 30. Thus, for each real-life scenario, a corresponding reference emotional state set 40 will indicate how a group of human observers would translate the outcome of that action in terms of emotion(s).

In the case where the emotional response engine to be trained is an ME, the human data acquisition processor 20 may be configured for reducing the human morality vectors determined for the human subjects 12 into a single reference human morality vector for each real-life scenario of the computer model 30. For example, the values of the reference human morality vector for each action can respectively be functions (e.g., an average or median) of the corresponding values in the multiple human emotional response vectors. That is, the first values contained in the human morality vectors may be averaged to yield the first value of the reference human emotional response vector, the second values contained in the human emotional response vectors may be averaged to yield the second value of the reference human morality vector, and so forth. Thus, for each action, a corresponding reference human morality vector will indicate how a group of human observers would translate the outcome of that action in terms of morality.

In the case where the emotional response engine to be trained is a KE, the human data acquisition processor 20 may be configured for reducing the human kindness levels determined for the human subjects 12 into a single reference human kindness level for each real-life scenario of the computer model 30. For example, the reference human kindness level for each action can respectively be a function (e.g., an average or median) of the multiple human kindness levels. Thus, for each action, a corresponding reference human kindness level will indicate how a group of human observers would translate the outcome of that action in terms of kindness.

The emotional response engine training processor 24 is configured for generating and storing an emotional response engine 38 (in the form of a neurome, described in U.S. Provisional Application 63/047,991 previously incorporated by reference) in the memory 22. The emotional response engine 38 is configured for predicting emotional state sets 42 in response to an input of the computer model of real-life scenarios 30 that may occur in the context of a range of use of an AI control system (e.g., operating or controlling autonomous cars).

The emotional response engine 38 may take the form of any suitable machine learning algorithm, which may provide a regression output and may contain various components and layers that can include but are not limited to: classical machine learning models such as support vector machines, random forests, or logistic regression, as well as modern deep learning models such as deep convolutional neural networks, attention-based networks, recurrent neural networks, or fully connected neural networks. The goal is for the emotional response engine 38 to accurately predict future data, i.e., by virtue of the emotional state sets output by the emotional response engine 38 in response to the input of the list of actions.

The emotional response engine 38 may be embodied in physical hardware, such as an Application Specific Integrated Circuit (ASIC), Field Programmable Gate Array (FPGA), Graphics Processing Unit (GPU), etc., to achieve very high-speed calculations in a moment of crisis. Physical hardware also decreases the possibility of software errors or changes to the algorithm. Encryption may also be used to verify the code, including hashing, bit checks, blockchain, or other security implemented either in software or hardware. Thus, encoding the emotional response engine 38 is physical hardware increases the number of possible actions analyzed in a given time, prevents tampering, and may be integrated with other hardware systems.

The emotional response engine training processor 24 is configured for training the emotional response engine 38 (which may start as a generic model of a human brain) on the computer model of real-life scenarios 30, such that the fully trained emotional response engine 38 predicts the collective emotional response of a particular group of humans, at least with respect to the same genre of computer model of real-life scenarios 30 on which the emotional response engine 38 has been trained.

Thus, the emotional response engine 38, once fully trained, may collectively emulate the brains of humans in that the emotional response engine 38 may predict the emotional states of humans in response to any real-life scenario that may occur in the context of the determined range of use of the AI control system in which the emotional response engine 38 will be subsequently used, even though such particular real-life scenario is not one of the real-life scenarios 30 on which the emotional response engine 38 has been trained. In this regard, the fully trained emotional response engine 38 collectively emulates the brains of humans in that it allows the emotional states of the humans to be predicted in response to new real-life scenarios that that the emotional response engine 38 has not previously experienced.

Thus, in response to outcomes to actions that are different from the computer model of real-life scenarios 30 (i.e., outcomes to actions) on which the emotional response engine 38 has been trained, the fully trained emotional response engine 38 may output emotional state sets 42 that are respectively predictive of emotional states of the human subjects 12 had these different outcomes to actions been presented to the human subjects 12.

To this end, the emotional response engine training processor 24 is further configured for updating the emotional response engine 38 via control signals 44 based on the computer model of real-life scenarios 30 and the emotional state sets 42 predicted by the emotional response engine 38 (e.g., the predicted human morality vectors if the emotional response engine 38 is an ME or the predicted human kindness level if the emotional response engine 38 is a KE).

The emotional response engine 38 may be trained by inputting the computer model of real-life scenarios 30, and updating the emotional response engine 38 via the control signals 44 in such a manner that the emotional state sets output by the emotional response engine 38 in response to input of the computer model of real-life scenarios 30 substantially match the reference emotional state sets acquired from the human subjects 12 by the human data acquisition processor 20.

In one embodiment, the emotional response engine training processor 24 is configured for respectively comparing the reference emotional state sets and the predicted emotional state sets 42 (e.g., comparing the reference human morality vectors and the predicted human morality vectors if the emotional response engine 38 is an ME or comparing the reference human kindness levels and the predicted human kindness levels if the emotional response engine 38 is a KE), generating at least one error signal based on the comparison, and updating the emotional response engine 38 via the control signals 44 based on the error signal(s).

It should be appreciated that although the human data acquisition processor 20 and the emotional response engine training processor 24 are illustrated as separate and distinct processors for purposes of clarity, the functionality (or any portions thereof) of the human data acquisition processor 20 and emotional response engine training processor 24 may be merged into a single processor. Furthermore, although each of the human data acquisition processor 20 and the emotional response engine training processor 24 may be configured as a single processor, the functionality of each of the human data acquisition processor 20 and the emotional response engine training processor 24 may be distributed amongst several processors. It should also be appreciated that those skilled in the art are familiar with the term “processor,” and that it may be implemented in software, firmware, hardware, or any suitable combination thereof.

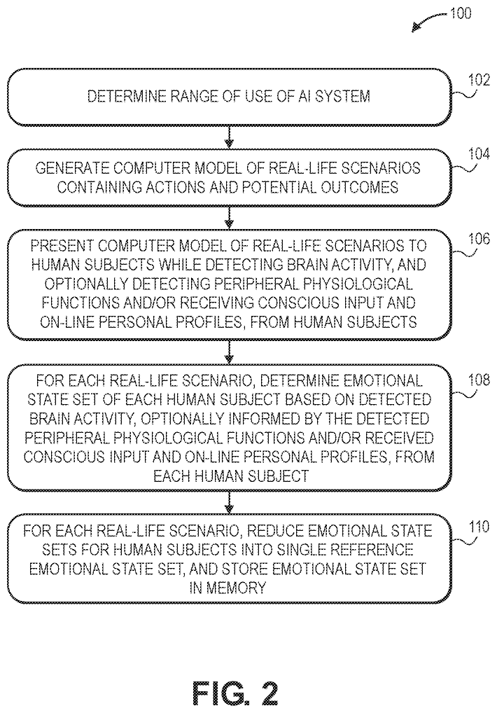

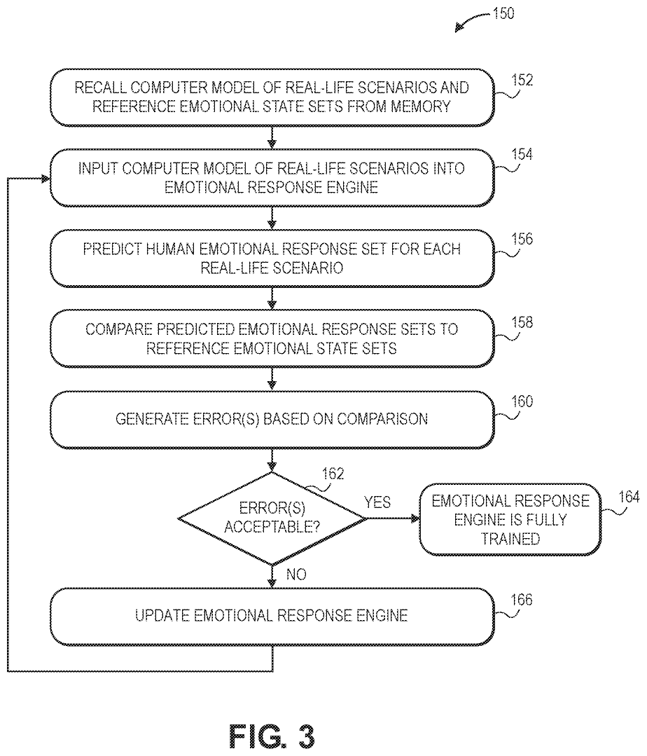

Having described the structure and function of the emotional response engine generation system 10, one exemplary method of operating the emotional response engine generation system 10 to train an emotional response engine 38 will now be described. This method can be divided into a human data acquisition method 100 for initially generating a large dataset of human brain activity related to observing the outcomes of actions (FIG. 2), and an emotional response engine training method 150 for subsequently training the emotional response engine 38 on the dataset of human brain activity generated by the method 100 (FIG. 3).

Referring to FIG. 2, the method 100 comprises determining the range of use of the AI control system (e.g., an AI control system for use in autonomous cars) (step 102), and generating and storing the computer model of a real-life scenarios 30 containing the actions and potential outcomes in memory (e.g., the memory 22) (step 104). Next, the computer model of a real-life scenarios 30 (e.g., demonstrating morality (or no morality) or demonstrating kindness (or no kindness) is presented to the human subjects 12 (e.g., via the human data acquisition processor 20 and UIs 16), while detecting the brain activity 32 of the human subjects 12 (e.g., via the non-invasive brain interface assemblies 18), and optionally detecting peripheral physiological functions 34 of the human subjects 12, e.g., heart rate, pupil size, respiratory rate, blood pressure, skin conductivity, etc. (e.g., via the peripheral sensors 26), and/or receiving conscious input 36 from the human subjects 12 (e.g., via the UIs 16) indicating one or more emotional states of the human subjects 12, and/or on-line personal profiles 28 of the human subjects 12 (step 106).

The human subjects 12 to which the computer model of a real-life scenarios 30 is presented may be from a very large population pool with diverse backgrounds and scenarios to limit or remove social, racial, educational, and gender related aspects. Furthermore, subject fatigue to evaluating a large number of emotional scenarios may result, so that a large number of human subjects may be necessary to generate a response pool. In the case where the emotional response engine 38 is an ME customized to a particular country or culture, the human subjects 12 may be from the same country where cultural differences affect moral decision making in humans.

Next, for each real-life scenario 30 presented to the human subjects 12, an emotional state set (e.g., joy, excitement, relaxation, surprise, fear, stress, anxiety, sadness, anger, disgust, contempt, contentment, calmness, approval, etc.) for each of the human subjects 12 is determined (via the emotional state determination processor 20) based on the detected brain activity 32, optionally informed by the detected peripheral physiological functions 34 of each human subject 12, conscious input 36 from each human subject 12, and/or personal profile 28 of each human subject 12 (step 108). As discussed above, the emotional state sets may take the form of human emotional state vectors in the case where the emotional response engine 38 to be trained is an ME or a human kindness levels in the case where the emotional response engine 38 to be trained is a KE.

Next, for each real-life scenario 30 presented to the human subjects 12, the determined emotional state sets for the human subjects 12 are reduced to a single reference emotional state set 40 (via the emotional state determination processor 20) representative of the collective emotional response of the human subjects 12 for each real-life scenario 30, and stored in the memory 22 (step 110). The reference emotional state set may take the form of a reference human emotional state vector in the case where the emotional response engine 38 to be trained is an ME or a reference human kindness level in the case where the emotional response engine 38 to be trained is a KE.

Thus, for each real-life scenario 30, a corresponding reference emotional state set 40 will indicate how a group of human observers would translate the real-life scenario 30 in terms of, e.g., morality in the case where the emotional response engine 38 is an ME or kindness in the case where the emotional response engine 38 is a KE.

As discussed above, using the emotional response engine training method 150 (FIG. 3), the emotional response engine 38 may be trained on the computer model of real-life scenarios 30 and reference emotional state sets 40 generated in the human data acquisition technique 100 illustrated above with respect to FIG. 2. To this end, and with reference to FIG. 3, the computer model of real-life scenarios 30 and reference emotional response sets 40 are recalled from memory (e.g., from the memory 22) (step 152). Next, the computer model of real-life scenarios 30 is input into the emotional response engine 38 (e.g., via the emotional response engine training processor 24) (step 154), such that the emotional response engine 38 predicts an emotional state set 42 for each real-life scenario 30 (step 156).

Next, the predicted emotional state sets 42 are respectively compared (e.g., via the emotional response engine training processor 24) to the reference emotional state sets 40 previously generated in the human data acquisition technique 100 illustrated above with respect to FIG. 2 (step 158), and generating one or more errors (e.g., via the emotional response engine training processor 24) based on the comparison (step 160).

In the case where the emotional response engine 38 to be trained is an ME, the predicted emotional state sets 42 (i.e. the predicted human morality vectors) may be respectively compared to the reference emotional state sets 40 (i.e., the reference human morality vectors). For example, the values of a predicted human emotional response vector may be respectively compared to the values of the corresponding reference human emotional response vector, and a function (e.g., an average or mean) of the errors between the respective values of predicted human emotional response vector and corresponding reference human emotional response vector can be computed.

In the case where the emotional response engine 38 to be trained is an KE, the predicted emotional state sets 42 (i.e. the predicted human kindness levels) may be respectively compared to the reference emotional state sets 40 (i.e., the reference human kindness levels).

Next, it is determined whether the error(s) (e.g., via the emotional response engine training processor 24) is acceptable (step 162). For example, the error(s) may be compared to one or more threshold values, and if the error(s) exceeds the threshold value(s), the error(s) may be determined to be unacceptable, and if the error(s) does not exceed the threshold value(s), the error(s) may be determined to be acceptable. In one method, a function of the errors (e.g., an average of the errors or a maximum of the errors) generated from the respective comparisons between the predicted emotional state sets 42 and the reference emotional state sets 40 may be averaged to yield a single error, which can then be compared to a single threshold value.

If the error(s) is acceptable, the emotional response engine 38 is deemed to be fully trained (step 164). If the error(s) is not acceptable, the emotional response engine 38 is updated (via the emotional response engine training processor 24) (step 166), and the method is repeated for the updated emotional response engine 38. The emotional response engine training processor 24 may subsequently be updated as new real-life scenarios are modeled or additional reference emotional data sets 40 are obtained.

Referring now to FIG. 4, one embodiment of an artificial intelligence (AI) system 50 that utilizes the emotional response engine 38 generated by the emotional response engine generation system 10 described above will be described. The emotional response engine 38, in this context, provides oversight for the AI control system 50 to reduce nonhuman or inhumane behaviors and actions in response to particular situations.

For example, if the AI control system 50 is a control system for an autonomous car, one particular situation may be a crisis situation in which the car is traveling at 60 mph on a road and approaching an intersection where a child is walking on a sidewalk as a dog runs into the street in the path of the car. The positive actions for the AI control system 50 to select in this crisis situation are to strike and kill the dog while not injuring the child, swerve away from the dog and hit and injury the child, or swerve into a telephone pole and injure the driver. Thus, the AI control system 50 must select between these three actions, all of which have negative outcomes. As another example, instead of a crisis situation, the AI control system 50 may have a primary objective of driving a passenger to an airport in time to make a flight, and may select any number of actions along the way to optimize this primary objective, including driving at the maximum speed, stopping for a dog crossing the street, driving through yellow lights, etc. As will be described in further detail below, the emotional response engine 38 aids the AI control system 50 to select the best action.

The AI control system 50 generally comprises memory 52, at least one sensor 54 (in this case, a plurality of sensors 54), an AI processor 56, and one or more actuators 58.

The memory 52 is configured for storing the emotional response engine 38 and a cost/reward function 60, and the sensor(s) 54 are configured for sensing an external environment of the AI control system 50 and outputting environment signals 62. The sensor(s) 54 may be, e.g., cameras, and the environmental signals 62 may be, e.g., video.

The AI processor 56 is configured for simulating different real-life scenarios 64 (i.e., actions and potential outcomes) that may occur in the context of the range of use of an AI control system (e.g., autonomous cars) in response to the environmental signals 52 output by the sensor(s) 54, as well as simulating typical (across many different human subjects) human emotional responses to the simulated real-life scenarios 64 by inputting each of the real-life scenarios 64 into the emotional response engine 38, such that the emotional response engine 38 respectively outputs a plurality of predicted emotional state sets 66. In the case where the emotional response engine 38 is an ME, the predicted emotional state sets 66 may be, e.g., predicted human emotional response vectors. In the case where the emotional response engine 38 is a KE, the predicted emotional state sets 66 may be, e.g., predicted human kindness levels.

The AI processor 56 is further configured for inputting the predicted emotional state sets 66 into a cost/reward function 60, such that the cost/reward function 60 outputs a plurality of scores 70 (e.g., morality scores in the case where the emotional response engine 38 is an ME or kindness scores in the case where the emotional response engine 38 is a KE) respectively for the real-life scenarios 64.

If the particular situation posed to the AI control system 50 is a crisis situation in which all of the actions have negative outcomes, each score 70 output by the cost/reward function 60 may simply be the average or sum of the human emotional response vector (in the case where the emotional response engine 38 is an ME) or a numerical value of the human kindness level (in the case where the emotional response engine 38 is a KE).

If the AI control system 50 has a primary objective unrelated to morality or kindness, and the performance of the AI control system 50 in a moral or kind manner is the secondary objective, the cost/reward function 60 may perform a tradeoff between achieving the primary objective and performing tasks in a moral or kind manner. For example, if the primary objective is to drive a passenger to the airport in time to make a flight, absent the emotional response engine 38, the AI control system 50 may perform actions to maximize the possibility of achieving this primary objective irrespective of whether any of the actions are immoral, including injuring a pedestrian. However, with the emotional response engine 38, the AI control system 50 weighs the pros and cons of being a little late to the airport and injuring a pedestrian. Thus, the AI control system 50 may risk being a little late to the airport if the risk of injuring a pedestrian becomes too great.

To this end, the cost/reward function 60 may take into account both the primary objective and a secondary objective (morality or kindness), e.g., by summing a value associated with the primary objective P and a value (e.g., average or sum of the human emotional response vector in the case where the emotional response engine 38 an ME or a numerical value of the kindness level in the case where the emotional response engine 38 is a KE) associated with the secondary objective S. The AI processor 56 is configured for dynamically varying the primary objective value P based on the probability that a particular action achieves the primary objective. For example, driving the car fast (beyond the posted legal speed limit) or running a yellow light will increase the primary objective value P, while driving slow or stopping at the yellow light will decrease the primary objective value P.

The AI processor 56 may dynamically weight the primary objective value P with a weighting value w1 based on the current performance of the primary objective. For example, if the performance of the primary objective decreases irrespective of the action that is selected (e.g., the chance that the flight will be missed increases due to traffic), the AI processor 56 may decrease the weighting value w1 in the case of a cost function) or increasing the weighting value w1 in the case of a reward function). In contrast, if the performance of the primary objective increases irrespective of the action that is selected (e.g., the chance that the flight will be missed decreases due to no traffic), the AI processor 56 may increase the weighting value w1 in the case of a cost function or decrease the weighting value w1 in the case of a reward function. Thus, the AI processor 56 may constantly change the weighting value w1 of the primary objective value P based on the risk of not achieving the primary objective (e.g., how late the passenger is to the airport).

The AI processor 56 may also weight the secondary objective value S with a weighting value w2 with a probability factor. In particular, the risk of an action can be defined as the product of the probability that the outcome of the action will occur and the severity of the occurrence (probability (p) multiplied by severity (s), (p*s)). For example, a human driver may decide to drive faster than the posted legal speed limit, because they assess the risk of being late for work lower than the risk of an accident. Thus, the AI processor 56 may need to provide a “risk assessment” that includes both the severity of the outcome, as well as the probability that the outcome might occur.

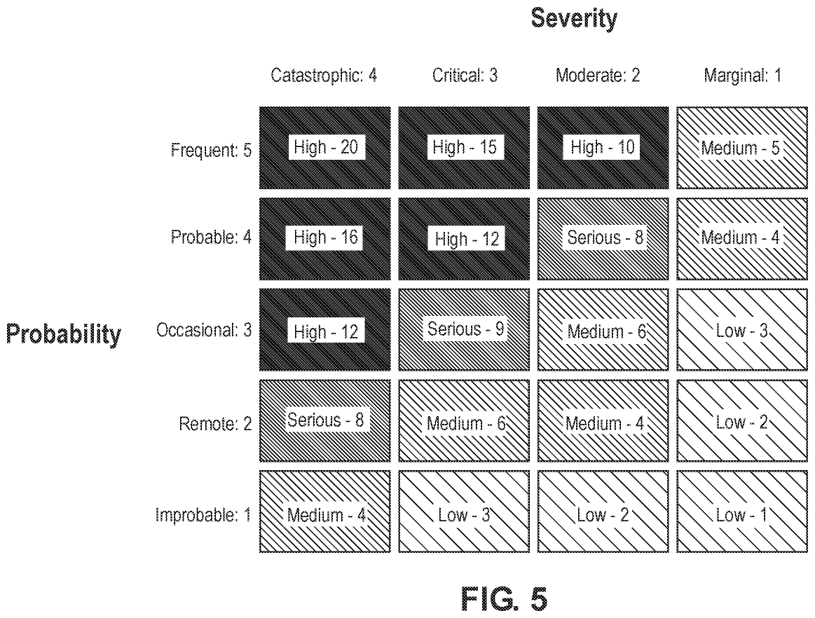

The severity of the occurrence may be ascertained from the corresponding predicted emotional state set 66 (i.e., it can be assumed that the more negative the predicted emotional state set 66, the more severe the outcome of the action is). As shown in FIG. 5, a probability versus severity matrix can be used to weight the predicted emotional state set 66 to yield a score 70. In particular, the severity of the outcome may be assigned different values ranging from low severity to high severity (e.g., marginal (1), moderate (2), critical (3), and catastrophic (4)), while the probability of the outcome may be assigned different values ranging from a low probability to a high probability (e.g., improbable (1), remote (2), occasional (3), probably (4), and frequent (5)). The severity of the outcome and the probability of the outcome may be multiplied, yielding values ranging from 1 to 20.

The primary objective value P and secondary objective value S may be weighted relative to each other, e.g., applying a weighting factor W to the primary objective value P and/or secondary objective value S. Thus, the cost/reward function 60 may output a score 70 in accordance with W*(w1*P)+w2*S.

In one embodiment, the weighting factor W may be set by the manufacturer in accordance with governmental regulations. In another embodiment, the weighting factor W may be manually adjusted (e.g., by the passenger) to tune the morality or kindness of the AI control system 50. For example, if the passenger is not risk adverse, the passenger may manually decrease the weighting factor W (within certain limits) in the case of a cost function or increase the weighting factor W (within certain limits in the case of a reward function), so that the AI control system 50 performs in a less moral or kind manner to increase the chance that the primary objective of the AI control system 50 will be achieved (e.g., getting to the airport in time to catch a flight). In contrast, if the passenger is risk adverse, the passenger may manually increase the weighting factor W in the case of a cost function or manually decrease the weighting factor W in the case of a reward function, so that the AI control system 50 performs in a more moral or kind manner to increase the chance that the AI control system 50 does not perform an immoral or unkind act (e.g., hitting or disrespecting a pedestrian).

The AI processor 56 is further configured for selecting one of the actions (one of the real-life scenarios 64) based on the scores 70 output by the cost/reward function 60, and performing the selection action. In the illustrated embodiment, the AI processor 56 selects the action with the best score 70. For a cost function, the best score 70 will be the lowest score, and for a reward function, the best score 70 will be the highest score.

The AI processor 56 is further configured for generating actuation signals 72 in accordance with the selected action, and sending the actuation signals 72 to the actuator(s) 58. The actuator(s) 58 are configured for performing the selected action.

For example, if the AI control system 50 is for use in the autonomous cars, the actuator(s) 58 may comprise, e.g., an accelerator, brake, steering mechanism, manual drive mode selection mechanism, etc., and the selected action may be changing the speed of the vehicle or changing a direction of the vehicle, or selection of manually driving the vehicle.

As such, the AI control system 50 incorporates a human emotion function (e.g., morality or kindness) into the decision-making process. The decision that achieves the optimum simulated human emotional response, while when possible also achieving the desired goal, is chosen. For example, a driving style that minimizes human anxiety (prediction of possible fatal injury for passengers and bystanders) is chosen that also gets the passenger to their destination in an acceptable on-time planned schedule.

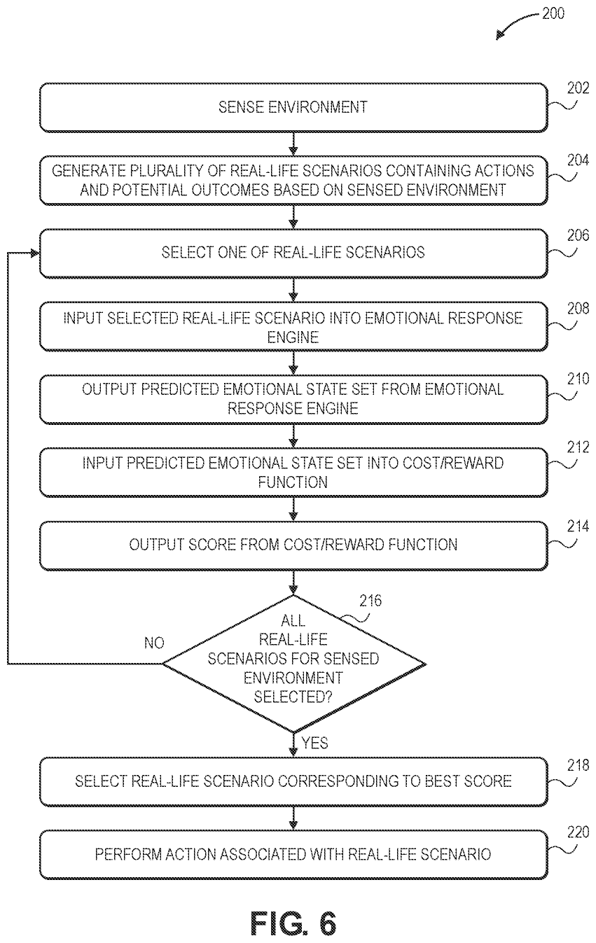

Having described the structure and function of the AI control system 50, one exemplary method 200 of operating the AI control system 50 will now be described with reference to FIG. 6.

The method 200 initially comprises presenting the AI control system 50 (FIG. 4) with a particular situation in which the AI control system 50 may make a decision between different real-life scenarios 64 (i.e., actions and associated outcomes). In particular, an environment is sensed (e.g., via the sensor(s) 54) (step 202), and a plurality of real-life scenarios 64 for the environment is generated based on the sensed environment (e.g., via the AI processor 56) (step 204). One of the real-life scenarios 64 is selected (step 206), and input into the emotional response engine 38 (e.g., via the AI processor 56) (step 208). A predicted emotional state set 66 is output from the emotional response engine 38 (e.g., a predicted emotional state vector in the case where the emotional response engine 38 is an ME and a predicted kindness level in the case where the emotional response engine 38 is a KE) (step 210).

The predicted emotional state set 66 is then input into the cost/reward function 60 (e.g., via the AI processor 56) (step 212), and a score 70 is output from the cost/reward function 60 (step 214). Next, it is determined whether all of the real-life scenarios 64 have been selected for the sensed environment and input into the emotional response engine 38 (e.g., via the AI processor 56) (step 216). If not all of the real-life scenarios 64 for the sensed environment have been selected and input into the emotional response engine 38, another one of the real-life scenarios 64 is selected (step 206), and the steps 208-216 are repeated for the newly selected real-life scenario 64. If all of the real-life scenarios 64 for the sensed environment have been selected and input into the emotional response engine 38, the real-life scenario 64 corresponding to the best score 70 is selected (e.g., via the AI processor 56) (step 218). The action associated with the selected real-life scenario 64 is then performed (e.g., via the actuator(s) 58) (step 220).

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed is:

1. A system for training an emotional response engine for use in an artificial intelligence (AI) system, comprising:
memory configured for storing the emotional response engine, wherein the emotional response engine is configured for predicting an emotional state set in response to an input of a real-life scenario that may occur in the context of a range of use of the AI control system;
at least one user interface (UI) configured for presenting the real-life scenario to each of a plurality of human subjects;
at least one non-invasive brain interface assembly configured for detecting brain activity of the plurality of human subjects in response to presenting the real-life scenario to each of the plurality of human subjects; and
at least one processor configured for determining a plurality of emotional state sets respectively for the plurality of human subjects based on the detected brain activity of the respective human subject, and updating the emotional response engine based on the predicted emotional state set and the plurality of determined emotional state sets.

2. The system of claim 1, wherein the at least one processor is configured for reducing the plurality of determined emotional state sets into a single reference emotional state set representative of a collective emotional response of the plurality of human subjects, comparing the single reference emotional state set and the predicted emotional state set, generating at least one error based on the comparison, and updating the emotional response engine based on the at least one error.

3. The system of claim 1, wherein the emotional response engine is a morality engine, the predicted emotional state set is a predicted human morality vector, the plurality of determined emotional state sets are a plurality of determined human morality vectors, and the at least one processor is configured for updating the morality engine based on the predicted human morality vector and the plurality of determined human morality vectors.

4. The system of claim 3, wherein the at least one processor is configured for deriving a single reference human morality vector from the plurality of determined human morality vectors, comparing the single reference human morality vector and the predicted human morality vector, generating at least one error based on the comparison, and updating the morality engine based on the at least one error.

5. The system of claim 1, wherein the emotional response engine is a kindness engine, the predicted emotional state set is a predicted kindness level, the plurality of determined emotional state sets are a plurality of determined kindness levels, and the at least one processor is configured for updating the kindness engine based on the predicted kindness level and the plurality of determined kindness levels.

6. The system of claim 5, wherein the at least one processor is configured for deriving a single reference human kindness level from the plurality of determined kindness levels, comparing the single reference human kindness level and the predicted kindness level, generating at least one error based on the comparison, and updating the kindness engine based on the at least one error.

7. A method of training an emotional response engine for use in an artificial intelligence (AI) system, comprising:
determining a range of use of the AI control system;
inputting a real-life scenario that may occur in the context of the AI control system into an emotional response engine;
outputting a predicted emotional state set from the emotional response engine in response to the input of the real-life scenario into the emotional response engine;
presenting the potential action outcome to each of a plurality of human subjects;
detecting brain activity of the plurality of human subjects in response to presenting the potential action outcome to each of the plurality of human subjects;
determining a plurality of emotional state sets respectively for the plurality of human subjects based on the detected brain activity of the plurality of human subjects; and
updating the emotional response engine based on the predicted emotional state set and the plurality of determined emotional state sets.

8. The method of claim 7, further comprising:
reducing the plurality of determined emotional state sets into a single reference emotional state set representative of a collective emotional response of the plurality of human subjects;
comparing the single reference emotional state set and the predicted emotional state set;
generating at least one error based on the comparison;
wherein the emotional response engine is updated based on the at least one error.

9. The method of claim 7, wherein the emotional response engine is a morality engine, the predicted emotional state set is a predicted human morality vector, the plurality of determined emotional state sets are a plurality of determined human morality vectors, and the morality engine is updated based on the predicted human morality vector and the plurality of determined emotional states.

10. The method of claim 9, further comprising:
deriving a single reference human morality vector from the plurality of determined human morality vectors;
comparing the single reference human morality vector and the predicted human morality vector;
generating at least one error based on the comparison; and
updating the morality engine based on the at least one error.

11. The method of claim 7, wherein the emotional response engine is a kindness engine, the predicted emotional state set is a predicted kindness level, the plurality of determined emotional state sets are a plurality of determined kindness levels, and the kindness engine is updated based on the predicted kindness level and the plurality of determined kindness levels.

12. The method of claim 11, further comprising:
deriving a single reference human kindness level from the plurality of determined kindness levels;
comparing the single reference human kindness level and the predicted kindness level;
generating at least one error based on the comparison; and
updating the kindness engine based on the at least one error.

13. An artificial intelligence (AI) control system, comprising:
memory configured for storing an emotional response engine;
at least one sensor configured for sensing an external environment of the artificial intelligence system;
at least one processor is configured for generating a plurality of real-life scenarios based on the sensed external environment, inputting each of the plurality of real-life scenarios into the emotional response engine, such that the emotional response engine respectively outputs a plurality of predicted emotional state sets, inputting the plurality of predicted emotional state sets into a cost function or a reward function, such that the cost function or reward function outputs a plurality of scores respectively for the plurality of real-life scenarios, selecting one of the plurality of real-life scenarios based on the plurality of scores; and
one or more actuators configured for performing an action associated with the selected real-life scenario.

14. The AI control system of claim 13, wherein the at least one processor is configured for selecting the real-life scenario corresponding to the best score of the plurality of scores.

15. The AI control system of claim 13, wherein the performed action comprises at least one of modifying a speed of a vehicle and changing a direction of the vehicle.

16. The AI control system of claim 13, wherein the emotional response engine is a morality engine, and the plurality of predicted emotional state sets are a plurality of predicted human emotional response vectors.

17. The AI control system of claim 13, wherein the emotional response engine is a kindness engine, and the plurality of predicted emotional state sets are a plurality of predicted human kindness levels.

18. The AI control system of claim 13, wherein the cost function or reward function comprises probabilities of outcomes associated with the plurality of real-life scenarios.

19. The AI control system of claim 13, wherein the at least one processor is configured for determining a level of performance of a primary objective of the AI control system, wherein the cost function or reward function comprises a weighting dependent on the determined performance level of the primary objective of the AI control system.

20. A method of operating an artificial intelligence (AI) control system, comprising:
sensing an external environment;
generating a plurality of real-life scenarios based on the sensed external environment;
inputting each of the plurality of real-life scenarios into an emotional response engine;
outputting a plurality of predicted emotional state sets from the emotional response engine;
inputting the plurality of predicted emotional state sets into a cost function or a reward function;
outputting a plurality of scores from the cost function or reward function respectively for the plurality of real-life scenarios;
selecting one of the plurality of real-life scenarios based on the plurality of scores; and
performing an action associated with the selected real-life scenario.

21. The method of claim 20, wherein the selected real-life scenario corresponds to the best score of the plurality of scores.

22. The method of claim 20, wherein the performed action comprises at least one of modifying a speed of a vehicle and changing a direction of the vehicle.

23. The method of claim 20, wherein the emotional response engine is a morality engine, and the plurality of predicted emotional state sets are a plurality of predicted human emotional response vectors.

24. The method of claim 20, wherein the emotional response engine is a kindness engine, and the plurality of predicted emotional state sets are a plurality of predicted human kindness levels.

25. The method of claim 20, wherein the cost function or reward function comprises probabilities of outcomes associated with the plurality of real-life scenarios.

26. The method of claim 20, further comprising determining a level of performance of a primary objective of the AI control system, wherein the cost function or reward function comprises a weighting dependent on the determined performance level of the primary objective of the AI control system.
